(19) United States
(12) Patent Application Publication
(10) Pub. No.: US 2026/0306842 A1
(43) Pub. Date: Oct. 8, 2026

(54) LONG PULSE WIDTH SPINAL CORD STIMULATION FOR TREATING DIABETIC NEUROPATHY, AND ASSOCIATED SYSTEMS AND METHODS

(71) Applicant: Nevro Corp., Redwood City, CA (US)

(72) Inventors: Kerry Bradley, Glendale, CA (US); Satinderpall Singh Pannu, Pleasanton, CA (US)

(21) Appl. No.: 19/481,958

(22) PCT Filed: May 9, 2024
(86) PCT No.: PCT/US2024/028654

§ 371 Date: Nov. 6, 2025

Related U.S. Application Data
(60) Provisional application No. 63/501,122, filed on May 9, 2023.

(51) Int. Cl. A61N 1/36; A61N 1/02; A61N 1/05

(52) U.S. Cl. CPC A61N 1/36062; A61N 1/025; A61N 1/36071; A61N 1/36103; A61N 1/36135; A61N 1/36175; A61N 1/0551

(57) ABSTRACT

The present technology provides systems and methods for delivering electrical stimulation having relatively long pulse widths to treat patient neuropathies, including diabetic neuropathy. For example, some embodiments include delivering a therapy signal having individual pulses with pulse widths of between about 5 milliseconds and 2 seconds. The therapy signals may restore sensation lost in association with the diabetic neuropathy and/or reduce pain or other abnormal sensations associated with the diabetic neuropathy.

### CROSS-REFERENCE TO RELATED APPLICATION(S)

The present application claims priority to U.S. Provisional Patent Application No. 63/501,122, filed May 9, 2023, the disclosure of which is incorporated by reference herein in its entirety.

### TECHNICAL FIELD

The present technology is directed towards spinal cord modulation for treating patient conditions, and associated systems and methods.

### BACKGROUND

An estimated 20 million people in the United States have some form of peripheral neuropathy, a condition that develops as a result of damage to the peripheral nervous system (“PNS”). The PNS is a vast communications network that connects the central nervous system (“CNS”) to the limbs and organs, essentially serving as a communication relay going back and forth between the brain and spinal cord with the rest of the body. Damage to the PNS interferes with this communication pathway, and symptoms can range from numbness or tingling, to pricking sensations or muscle weakness. Peripheral neuropathy has been conventionally treated with medication, injection therapy, physical therapy, surgery, and light. More recently, diabetic peripheral neuropathy has been treated by applying a surface electrical stimulation at a specified frequency to the muscles and nerves. Most treatments are designed to treat the underlying cause and/or manifestations of the neuropathy, but in many cases, the cause of the neuropathy is unknown or effective treatments for the cause and manifestations may not exist. Accordingly, there is a need for systems and methods for treating peripheral neuropathy.

### BRIEF DESCRIPTION OF THE DRAWINGS

FIG. 1A is a partially schematic illustration of an implantable spinal cord modulation system positioned at a patient's spine to deliver therapeutic signals in accordance with some embodiments of the present technology.

FIG. 1B is a partially schematic, cross-sectional illustration of a patient's spine, illustrating representative locations for implanted lead bodies in accordance with some embodiments of the present technology.

FIG. 2 is a schematic illustration of a representative lead body suitable for providing modulation to a patient in accordance with several embodiments of the present technology.

FIGS. 3A-3H illustrate representative wave forms associated with therapy signals applied to patients in accordance with particular embodiments of the present technology.

FIG. 4 is a flow diagram illustrating a method for treating a patient in accordance with embodiments of the present technology.

FIG. 5A is a flow diagram illustrating a method for determining a maximum amplitude for a waveform having a given pulse width in accordance with embodiments of the present technology, and FIG. 5B is a series of scope traces for determining the maximum amplitude for a waveform having a given pulse width in accordance with the method shown in FIG. 5A.

### DETAILED DESCRIPTION

The present technology is generally directed to neurostimulation for the treatment of peripheral neuropathies such as diabetic neuropathy. In some embodiments, the present technology provides therapy signals having relatively long pulse widths, such as between about 5 milliseconds and about 2 seconds. In some embodiments, the therapy signals further include offset high frequency pulses and/or bursts of high frequency pulses occurring during the relatively long pulse widths. For example, the therapy signal can have a base component having a non-zero amplitude and a pulse width in a pulse width range of from about 5 milliseconds to about 2 seconds, and a high frequency component including high frequency pulses having a frequency in a frequency range of from about 1.2 kHz to about 100 kHz and occurring from the non-zero amplitude of the base component. Without being bound by theory, therapy signals in accordance with the present technology are expected to advantageously reduce one or more symptoms of the patient's neuropathy, such as pain, sensorimotor deficits, numbness, and/or abnormal sensations.

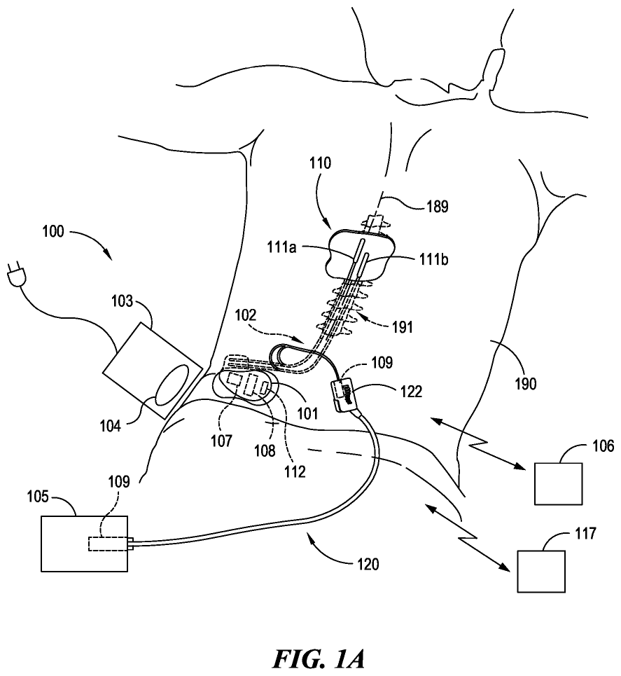

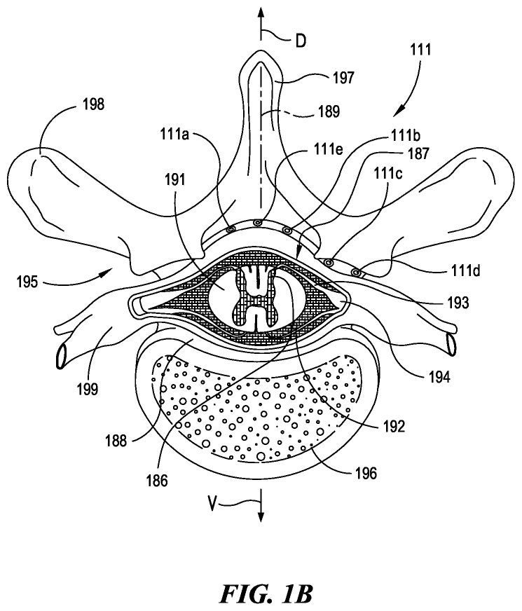

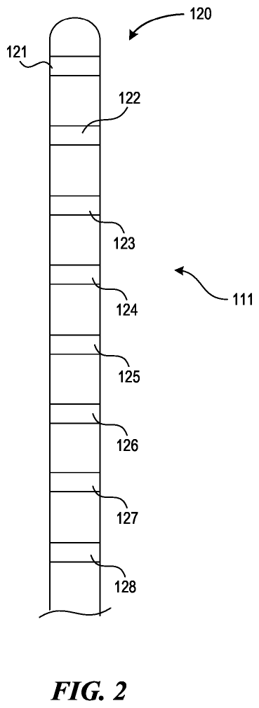

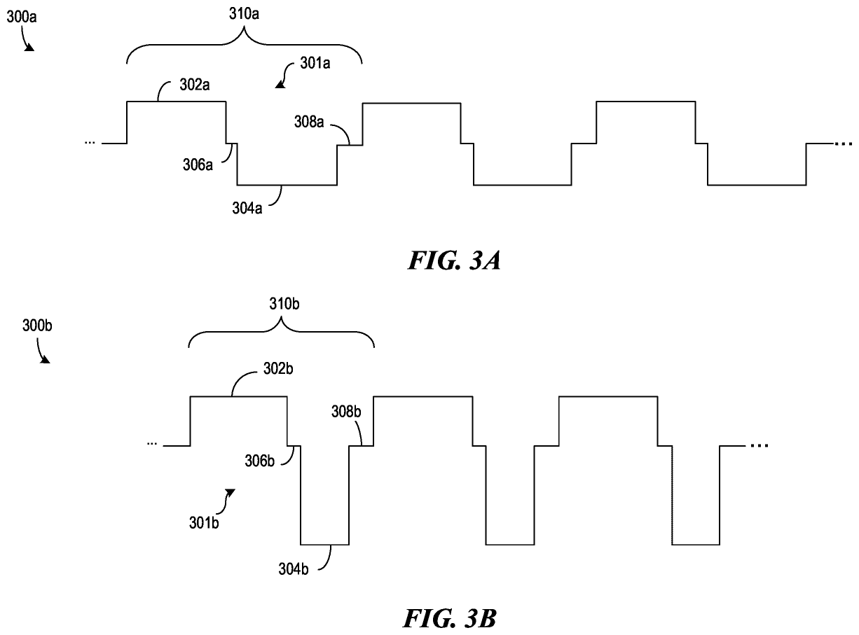

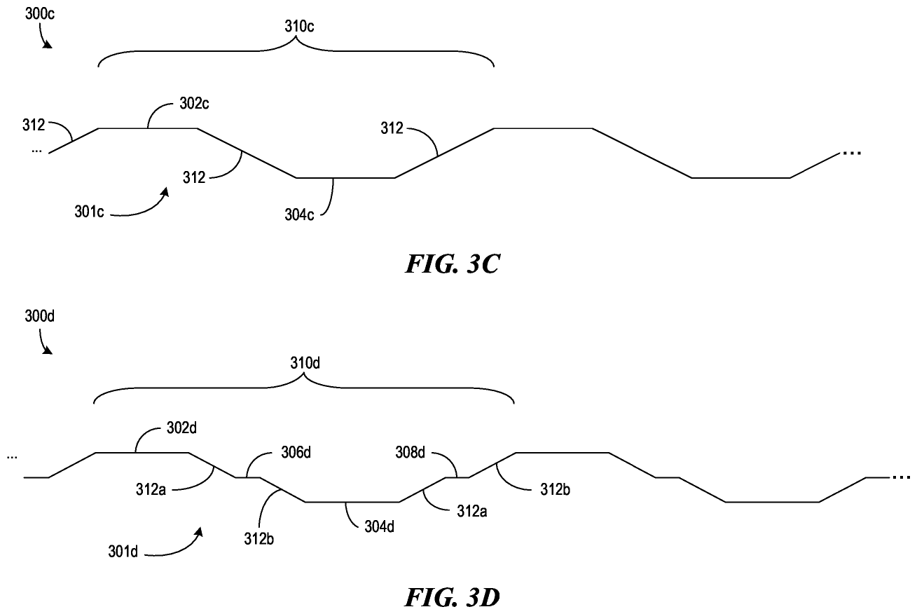

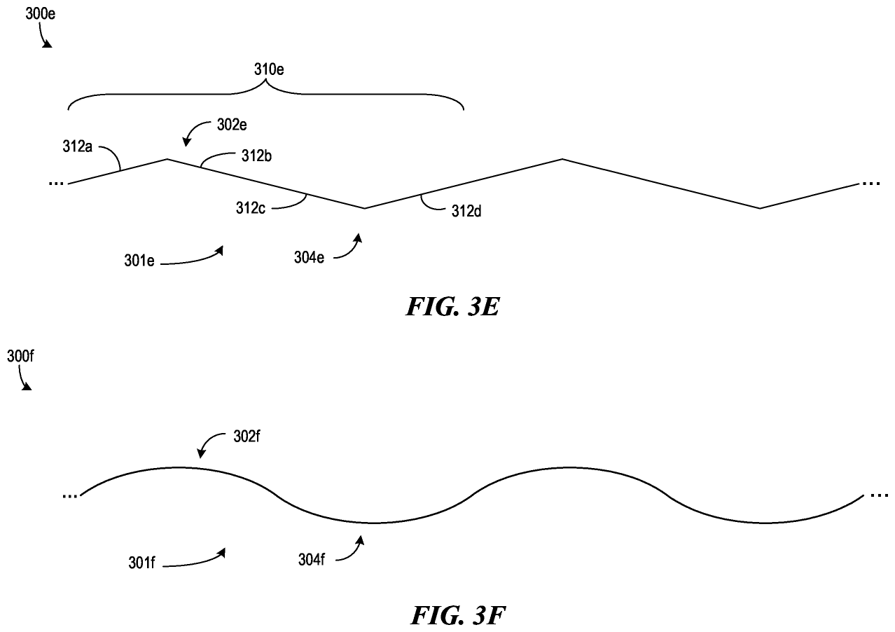

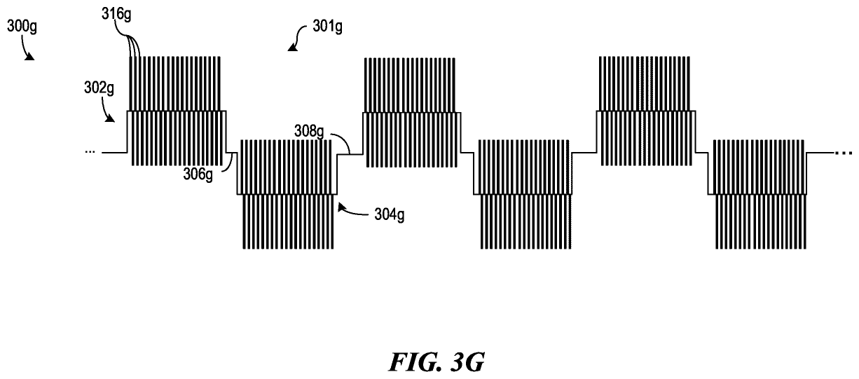

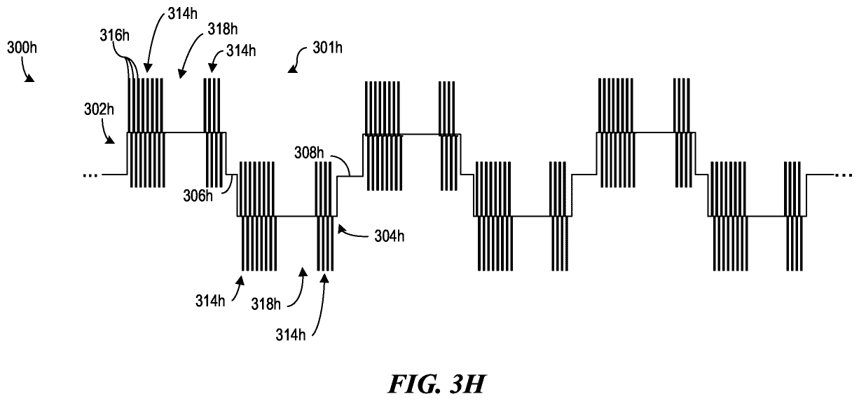

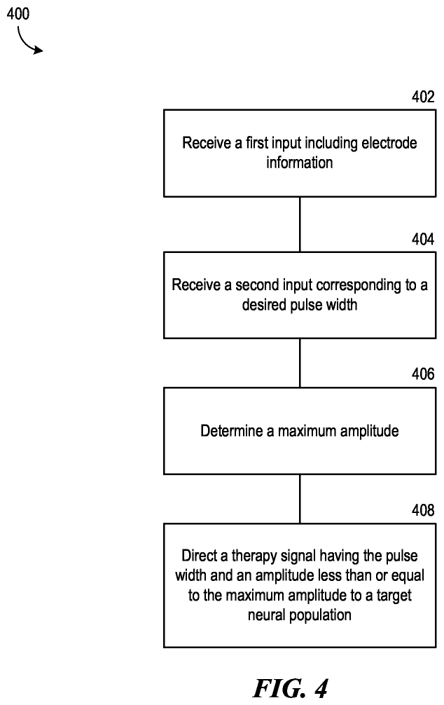

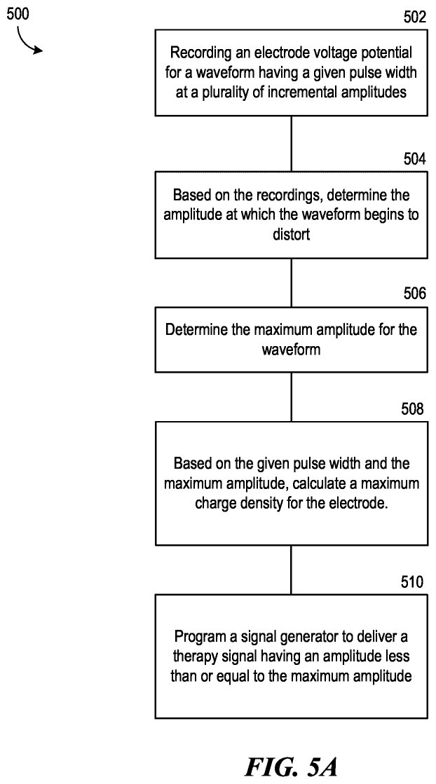

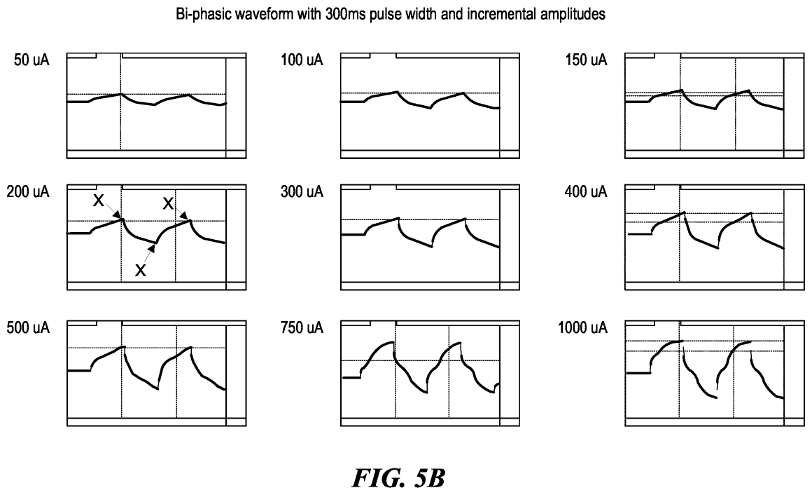

Definitions of selected terms are provided under Heading 1.0 (“Definitions”). General aspects of the present technology are described below under Heading 2.0 (“Overview of Present Technology”). Representative treatment systems and their characteristics are described under Heading 3.0 (“System Characteristics”) with reference to FIGS. 1A, 1B and 2. Representative electrical signals for treating patients are described under Heading 4.0 (“Representative Electrical Signals”) with reference to FIGS. 3A-3H. Representative methods for identifying suitable amplitudes for use with the electrical therapy signals described under Heading 5.0 (“Representative Methods for Determining Suitable Amplitudes of Electrical Signals Having Long Pulse Widths”) with reference to FIGS. 4-5B. Representative examples are described under Heading 6.0 (“Representative Examples”). The headings provided herein are for convenience only and do not interpret the scope or meaning of the claimed present technology.

### 1.0 DEFINITIONS

Unless otherwise stated, the terms “generally,” “about,” and “approximately” refer to values within 10% of a stated value. For example, the use of the term “about 100” refers to a range of 90 to 110, inclusive. In instances where relative terminology is used in reference to something that does not include a numerical value, the terms are given their ordinary meaning to one skilled in the art.

As used herein, the terms “therapy signal,” “electrical therapy signal,” “electrical signal,” “neuromodulation signal,” “signal,” and other associated terms are used interchangeably and generally refer to an electrical signal that can be characterized by one or more parameters, such as pulse width, amplitude, and/or frequency.

As used herein, and unless otherwise noted, the terms “modulate,” “modulation,” “stimulate,” and “stimulation” refer generally to signals that have an inhibitory, excitatory, and/or other effect on a target neural population. Accordingly, a spinal cord “stimulator” can have an inhibitory effect on certain neural populations. Moreover, the use of the terms “suppress” and “inhibit” in relation to a therapy signal's effect on a neuron refers to a reduction in the neuron's firing rate relative to the neuron's firing rate in the absence of the therapy signal, and does not necessarily refer to a complete elimination of action potentials in the neuron.

As used herein, “proximate a spinal cord region” refers to the placement of a signal delivery element such that it can deliver electrical stimulation to a neural population associated with the spinal cord or associated nervous system structures. For example, “proximate a spinal cord region” includes, but is not limited to, the relative lead positions described and shown in FIG. 1B, as well as other positions not expressly described herein.

As used herein, the term “pulse width” refers to the width of any phase of a repeating pulse, such as the portion of a pulse at a given polarity, unless explicitly described otherwise. For example, the use of the term pulse width with respect to a signal having bi-phasic pulses can refer to the duration of an anodic pulse phase or a cathodic pulse phase. The use of the term pulse width with respect to a signal having monophasic pulses can refer to the duration of the monophasic pulse phase.

### 2.0 OVERVIEW OF THE PRESENT TECHNOLOGY

Many patients with peripheral neuropathies experience a complex combination of symptoms that can include sensation loss (e.g., numbness, especially in the patient's extremities), pain (e.g., hyperalgesia, burning sensations, especially in the patient's extremities, etc.), other abnormal sensations (e.g., dysesthesia, tingling, paresthesia, etc.), impaired proprioception, and the like. The present technology is directed generally to spinal cord modulation and associated systems and methods for treating peripheral neuropathies, including by reducing, treating, improving, and/or alleviating one or more symptoms associated with the patient's peripheral neuropathy. For example, the spinal cord stimulation therapies described herein may restore lost sensation, improve proprioception, and/or reduce pain and/or other abnormal sensations associated with the patient's neuropathy. The spinal cord stimulation therapies described herein can be used to address a variety of peripheral neuropathies, including diabetic neuropathy (e.g., painful diabetic neuropathy, painless diabetic neuropathy, etc.), peripheral polyneuropathy, chemotherapy induced neuropathy, idiopathic neuropathy, or other neuropathies.

In some embodiments, representative techniques include applying a therapy signal having a relatively long pulse width of from about 5 milliseconds to about 2 seconds to a spinal cord region of a patient. In some embodiments, the therapy signals further include offset high frequency pulses and/or bursts of high frequency pulses occurring during the relatively long pulse widths. The therapy signal can be applied at an amplitude that is below the activation threshold of neurons adjacent the signal delivery element. The use of therapy signals in accordance with the present technology is expected to advantageously address one or more physiologic factors contributing to patient pain, loss of sensation, and/or abnormal sensations.

As set forth above, applying electrical signals having a pulse width between about 5 milliseconds and about 2 seconds to a patient's spinal cord region may restore sensation that was lost in association with the patient's peripheral neuropathy. For example, the electrical signals described herein are expected to reduce numbness in a patient's feet, legs, hands, and/or arms, amongst other locations.

Without intending to be bound by theory, one potential mechanism of action that may underlie the sensory restoration induced by electrical signals having relatively long pulse widths includes the electrical signals boosting/improving transmission of afferent neural signals traveling along the spinal cord. Many patients suffering from peripheral neuropathies have loss of, and/or damage to, neural receptors and nerve axons in the peripheral nervous system (e.g., downregulation of receptors, demyelination of axons, cell death, etc.). This may be in part due to loss of blood flow to the peripheral neurons and/or other disease-related factors. Regardless of the etiology, the damaged or malfunctioning peripheral nerves may generate a “weakened” signal in response to sensory stimuli, which in turn may not be properly transmitted to the patient's sensory cortex. The patient thus experiences reduced sensation or even numbness. However, the electrical stimulation described herein may enhance transmission of afferent signals generated in response to a sensory input to improve patient perception of sensory inputs.

As a first example, the electrical signals described herein may increase the firing capability and responsiveness of certain post-synaptic dorsal column neurons in the spinal cord (e.g., by operating as a “membrane stabilizer”). Such neurons may receive sensory inputs from peripheral nerves and transmit the sensory inputs (including both non-painful sensory information, such as pressure, touch, joint position, muscle tension, muscle stretch, etc., and painful sensory information) to the sensory cortex. Changing the excitability of these neurons may therefore increase the strength of the afferent signal that reaches the patient's sensory cortex, and increase the patient's perception of the sensory stimulus.

As another example, the fidelity of weakened afferent signals arriving from the periphery may be dampened due to ‘noise’ present in diseased hyperexcitable spinal cord neurons (e.g., ectopic activity, reduced thresholds in uncorrelated spinal circuitry causing aberrant firing patterns, etc.), further altering the transmission of non-painful sensory information to the sensory cortex. The electrical signals described herein may provide selective inhibition of these hyperexcitable spinal circuits responsible for abnormal transmission of signals to the brain, and allow the weaker signals received from the periphery to be sent to the thalamus and sensory cortex with better accuracy.

As yet another example, the electrical signals described herein may increase the excitability and/or responsiveness of the peripheral neurons conveying sensory information to the spinal cord, which in turn is expected to increase the strength of the afferent signal that reaches the patient's sensory cortex, and increase the patient's perception of the sensory stimulus. As one skilled in the art will appreciate from the disclosure herein, the foregoing mechanisms are provided by way of example only, and the electrical signals described herein may act to restore sensation through other mechanisms of action, in addition to or in lieu of the mechanisms described above.

Applying electrical signals having a pulse width of from about 5 milliseconds to about 2 seconds to a patient's spinal cord may also reduce pain and other abnormal sensations associated with the patient's peripheral neuropathy. For example, the electrical signals described herein are expected to reduce pain in a patient's feet, legs, hands, and/or arms, amongst other locations. As set forth above, the electrical signals described herein may improve the patient's ability to detect sensory inputs in addition to reducing patient pain. In this way, the electrical signals may improve the patient's ability to detect painful stimuli, such as a pin prick (e.g., improve a patient's nociception). However, even though the electrical signals may normalize a patient's nociception, the electrical signals described herein may nevertheless also reduce pain associated with the patient's peripheral neuropathy. For example, the electrical signals may reduce the patient's dysesthesia, allodynia, or other abnormal painful sensations associated with the patient's neuropathy.

Without intending to be bound by theory, one potential mechanism of action that may underlie the pain reduction induced using electrical signals having relatively long pulse widths includes the electrical signals directly and/or indirectly suppressing specific neurons that transmit pain signals to a patient's pain perception centers. For example, the electrical signals described herein may directly suppress neurons in the superficial dorsal horn pain circuits (e.g., nociceptive-specific and/or wide dynamic range neurons). This may cause neural membrane channels of such neurons to enter a net inactive state that prevents the neurons from firing, and therefore reduces and/or prevents transmission of painful afferent neural signals from reaching the patient's sensory cortex. The foregoing mechanism is provided by way of example only, and the electrical signals described herein may act to reduce pain through other mechanisms of action, in addition to or in lieu of the mechanism described above.

Specific details of certain embodiments of the disclosure are described below with reference to methods for modulating one or more target neural populations (e.g., nerves) or sites of a patient, and associated implantable structures for providing the modulation. Although selected embodiments are described below with reference to modulating the dorsal column, dorsal horn, dorsal root, dorsal root entry zone, and/or other particular regions of the spinal column to control pain and/or restore sensation, the modulation may in some instances be directed to other neurological structures and/or target neural populations of the spinal cord and/or other neurological tissues. Some embodiments can have configurations, components or procedures different than those described in this section, and other embodiments may eliminate particular components or procedures. A person of ordinary skill in the relevant art, therefore, will understand that the present disclosure may include other embodiments with additional elements, and/or may include other embodiments without several of the features shown and described below with reference to FIGS. 1A-5B.

### 3.0 SYSTEM CHARACTERISTICS

FIG. 1A schematically illustrates a representative patient therapy system 100 for treating a patient's motor and/or other functioning, arranged relative to the general anatomy of the patient's spinal column 191. The system 100 can include a signal generator 101 (e.g., an implanted or implantable pulse generator or IPG), which may be implanted subcutaneously within a patient 190 and coupled to one or more signal delivery elements or devices 110. The signal delivery elements or devices 110 may be implanted within the patient 190, at or off the patient's spinal cord midline 189. The signal delivery elements 110 carry features for delivering therapy to the patient 190 after implantation. The signal generator 101 can be connected directly to the signal delivery devices 110, or it can be coupled to the signal delivery devices 110 via a signal link, e.g., a lead extension 102. In some embodiments, the signal delivery devices 110 can include one or more elongated lead(s) or lead body or bodies 111 (identified individually as a first lead 111a and a second lead 111b). As used herein, the terms signal delivery device, signal delivery element, lead, and/or lead body include any of a number of suitable substrates and/or supporting members that carry electrodes/devices for providing therapy signals to the patient 190. For example, the lead or leads 111 can include one or more electrodes or electrical contacts that deliver electrical signals into the patient's tissue, e.g., to provide for therapeutic relief. In some embodiments, the signal delivery elements 110 can include structures other than a lead body (e.g., a paddle) that also deliver electrical signals and/or other types of signals to the patient 190, e.g., as disclosed in U.S. Patent Application Publication No. 2018/0256892, incorporated herein by reference in its entirety. For example, paddles may be more suitable for patients with spinal cord injuries that result in scarring or other tissue damage that impedes cylindrical leads.

In some embodiments, one signal delivery device may be implanted on one side of the spinal cord midline 189, and a second signal delivery device may be implanted on the other side of the spinal cord midline 189. For example, the first and second leads 111a, 111b shown in FIG. 1A may be positioned just off the spinal cord midline 189 (e.g., about 1 mm offset) in opposing lateral directions so that the two leads 111a, 111b are spaced apart from each other by about 2 mm. In some embodiments, the leads 111 may be implanted at a vertebral level ranging from, for example, about T4 to about T12. In some embodiments, one or more signal delivery devices can be implanted at other vertebral levels, e.g., as disclosed in U.S. Pat. No. 9,327,121, incorporated herein by reference in its entirety.

The signal generator 101 can transmit signals (e.g., electrical signals) to the signal delivery elements 110 that excite, inhibit, downregulate, and/or suppress target nerves. The signal generator 101 can include a machine-readable (e.g., computer-readable or controller-readable) medium containing instructions for generating and transmitting suitable therapy signals, such as those described below with respect to FIGS. 3A-3D. The signal generator 101 and/or other elements of the system 100 can include one or more processor(s) 107, memory unit(s) 108, and/or input/output device(s) 112. Accordingly, the process of providing modulation signals, providing guidance information for positioning the signal delivery devices 110, establishing battery charging and/or discharging parameters, and/or executing other associated functions can be performed by computer-executable instructions contained by, on, or in computer-readable media located at the pulse generator 101 and/or other system components. Further, the pulse generator 101 and/or other system components may include dedicated hardware, firmware, and/or software for executing computer-executable instructions that, when executed, perform any one or more methods, processes, and/or sub-processes described in the materials incorporated herein by reference. The dedicated hardware, firmware, and/or software also serve as “means for” performing the methods, processes, and/or sub-processes described herein. The signal generator 101 can also include multiple portions, elements, and/or subsystems (e.g., for directing signals in accordance with multiple signal delivery parameters), carried in a single housing, as shown in FIG. 1A, or in multiple housings. For example, the signal generator can include some components that are implanted (e.g., a circuit that directs signals to the signal delivery device 110), and some that are not (e.g., a power source). The computer-executable instructions can be contained on one or more media that are implanted within the patient and/or positioned external to the patient, depending on the embodiment.

The signal generator 101 can also receive and respond to an input signal received from one or more sources. The input signals can direct or influence the manner in which the therapy, charging, and/or process instructions are selected, executed, updated, and/or otherwise performed. The input signals can be received from one or more sensors (e.g., an input device 112 shown schematically in FIG. 1A for purposes of illustration) that are carried by the signal generator 101 and/or distributed outside the signal generator 101 (e.g., at other patient locations) while still communicating with the signal generator 101. The sensors and/or other input devices 112 can provide inputs that depend on or reflect patient state (e.g., patient position, patient posture, and/or patient activity level), and/or inputs that are patient-independent (e.g., time). Still further details are included in U.S. Pat. No. 8,355,797, incorporated herein by reference in its entirety.

In some embodiments, the signal generator 101 and/or signal delivery devices 110 can obtain power to generate the therapy signals from an external power source 103. For example, the external power source 103 can by-pass an implanted signal generator and generate a therapy signal directly at the signal delivery devices 110 (or via signal relay components). The external power source 103 can transmit power to the implanted signal generator 101 and/or directly to the signal delivery devices 110 using electromagnetic induction (e.g., RF signals). For example, the external power source 103 can include an external coil 104 that communicates with a corresponding internal coil (not shown) within the implantable signal generator 101, signal delivery devices 110, and/or a power relay component (not shown). The external power source 103 can be portable for ease of use. In embodiments without the implanted signal generator 101 and in which the external power source 103 causes a therapy signal to be generated directly at the signal delivery devices 110 or at an implanted relay device (not shown), the external power source 103 can be a wearable device that the patient wears while receiving therapy. In such embodiments, the patient only receives stimulation therapy while the wearable device is placed in an active state and is being worn by the patient. Such embodiments are generally less invasive because they generally do not require an implanted signal generator 101, nor an implanted power storage device.

In some embodiments, the signal generator 101 can obtain the power to generate therapy signals from an internal power source, in addition to or in lieu of the external power source 103. For example, the implanted signal generator 101 can include a non-rechargeable battery (e.g., a primary cell) or a rechargeable battery (e.g., a secondary cell) to provide such power. When the internal power source includes a rechargeable battery, the external power source 103 can be used to recharge the battery. The external power source 103 can in turn be recharged from a suitable power source (e.g., conventional wall power).

During at least some procedures, an external stimulator or trial modulator 105 can be coupled to the signal delivery elements 110 during an initial procedure, prior to implanting the signal generator 101. For example, a practitioner (e.g., a physician and/or a company representative) can use the trial modulator 105 to vary the modulation parameters provided to the signal delivery elements 110 in real time, and select optimal or particularly efficacious parameters. These parameters can include the location from which the electrical signals are emitted, as well as the characteristics of the electrical signals provided to the signal delivery devices 110. In some embodiments, input is collected via the external stimulator or trial modulator 105 and can be used by the clinician to help determine what parameters to vary. In a typical process, the practitioner uses a wireless connection or cable assembly 120 to temporarily connect the trial modulator 105 to the signal delivery device 110. The practitioner can test the efficacy of the signal delivery devices 110 in an initial position. The practitioner can then disconnect the cable assembly 120 if needed (e.g., at a connector 122), reposition the signal delivery devices 110, and reapply the electrical signals. This process can be performed iteratively until the practitioner obtains the desired position for the signal delivery devices 110. Optionally, the practitioner may move the partially implanted signal delivery devices 110 without disconnecting the cable assembly 120. Furthermore, in some embodiments, the iterative process of repositioning the signal delivery devices 110 and/or varying the therapy parameters may not be performed.

The signal generator 101, the lead extension 102, the trial modulator 105 and/or the connector 122 can each include a receiving element 109. Accordingly, the receiving elements 109 can be patient implantable elements, or the receiving elements 109 can be integral with an external patient treatment element, device or component (e.g., the trial modulator 105 and/or the connector 122). The receiving elements 109 can be configured to facilitate a simple coupling and decoupling procedure between the signal delivery devices 110, the lead extension 102, the pulse generator 101, the trial modulator 105 and/or the connector 122. The receiving elements 109 can be at least generally similar in structure and function to those described in U.S. Patent Application Publication No. 2011/0071593, incorporated by reference herein in its entirety.

After the signal delivery elements 110 are implanted, the patient 190 can receive therapy via signals generated by the trial modulator 105, generally for a limited period of time. During this time, the patient wears the trial modulator 105 outside the body. Assuming the trial therapy is effective or shows the promise of being effective, the practitioner then replaces the trial modulator 105 with the implanted signal generator 101, and programs the signal generator 101 with therapy programs selected based on the experience gained during the trial period. Optionally, the practitioner can also replace the signal delivery elements 110. In still further embodiments, the signal generator 101 can be implanted without first undergoing a trial period. Once the implantable signal generator 101 has been positioned within the patient 190, the therapy programs provided by the signal generator 101 can still be updated remotely via a wireless physician's programmer 117 (e.g., a physician's laptop, a physician's remote or remote device, etc.) and/or a wireless patient programmer 106 (e.g., a patient's laptop, patient's remote or remote device, etc.). Generally, the patient 190 has control over fewer parameters than does the practitioner. For example, the capability of the patient programmer 106 may be limited to starting and/or stopping the signal generator 101, selecting a pre-programmed therapy option, and/or adjusting the signal amplitude within a present amplitude range. The patient programmer 106 may be configured to accept inputs corresponding to pain relief, motor functioning and/or other variables, such as medication use. Accordingly, more generally, embodiments of the present technology include receiving patient feedback, via a sensor, that is indicative of, or otherwise corresponds to, the patient's response to the signal. Feedback includes, but is not limited to, motor, sensory, and verbal feedback. In response to the patient feedback, one or more signal parameters can be adjusted, such as frequency, pulse width, amplitude, or delivery location. In some embodiments, the patient programmer can be a network connected handheld computing device such as a smartphone, which can include a patient app that provides various functions such as remote programming, therapy selection, therapy tracking information such as current pain score or level of sensory restoration for use by the clinician and/or software in the app to optimize therapy.

FIG. 1B is a cross-sectional illustration of the spinal cord 191 and an adjacent vertebra 195 (based generally on information from Crossman and Neary, “Neuroanatomy,” 1995 (published by Churchill Livingstone)), along with multiple leads 111 (shown as leads 111a-111e) implanted at representative locations. For purposes of illustration, multiple leads 111 are shown in FIG. 1B implanted in a single patient. In addition, for purposes of illustration, the leads 111 are shown as elongated leads however, leads 111 can be paddle leads. In actual use, any given patient will likely receive fewer than all the leads 111 shown in FIG. 1B.

The spinal cord 191 is situated within a vertebral foramen 188, between a ventrally located ventral body 196 and a dorsally located transverse process 198 and spinous process 197. Arrows V and D identify the ventral and dorsal directions, respectively. The spinal cord 191 itself is located within the dura mater 199, which also surrounds portions of the nerves exiting the spinal cord 191, including the ventral roots 192, dorsal roots 193, and dorsal root ganglia 194. The dorsal roots 193 enter the spinal cord 191 at the dorsal root entry region 187, and communicate with dorsal horn neurons located at the dorsal horn 186. In some embodiments, the first and second leads 111a, 111b are positioned just off the spinal cord midline 189 (e.g., about 1 mm offset) in opposing lateral directions so that the two leads 111a, 111b are spaced apart from each other by about 2 mm, as discussed above. In some embodiments, a lead or pairs of leads can be positioned at other locations, e.g., toward the outer edge of the dorsal root entry portion 187 as shown by a third lead 111c, or at the dorsal root ganglia 194, as shown by a fourth lead 111d, or approximately at the spinal cord midline 189, as shown by a fifth lead 111e.

In some embodiments the devices and systems of the present technology include features other than those described herein. For example, one lead 111 to six leads 111 can be positioned generally end-to-end at or near the patient's midline M and span vertebral levels from about T4 to about T12. In some embodiments, two, three, or four leads 111 are positioned end-to-end at or near the patient's midline from T4 to T12. In some embodiments, the leads 111 and/or other signal delivery devices can have locations other than those expressly shown herein. For example, one or more signal delivery devices can be positioned at the dorsal side of the spinal cord 191. In addition, the devices and systems of the present technology can include more than one internal stimulator and/or more than one external stimulator that can be configured for wireless stimulation, such as by using electromagnetic waves.

Several aspects of the technology are embodied in computing devices, e.g., programmed/programmable pulse generators, controllers and/or other devices. The computing devices on/in which the described technology can be implemented may include one or more central processing units, memory, input devices (e.g., input ports), output devices (e.g., display devices), storage devices, and network devices (e.g., network interfaces). The memory and storage devices are computer-readable media that may store instructions that implement the technology. In some embodiments, the computer readable media are tangible media. In some embodiments, the data structures and message structures may be stored or transmitted via an intangible data transmission medium, such as a signal on a communications link. Various suitable communications links may be used, including but not limited to a local area network and/or a wide-area network.

FIG. 2 is a partially schematic illustration of a representative lead body 111 that may be used to apply modulation to a patient in accordance with any of the foregoing embodiments. In general, the lead body 111 includes a multitude of electrodes or contacts 120. When the lead body 111 has a circular cross-sectional shape, as shown in FIG. 2, the contacts 120 can have a generally ring-type shape and can be spaced apart axially along the length of the lead body 111. In a particular embodiment, the lead body 111 can include eight contacts 120, identified individually as first, second, third . . . eighth contacts 121, 122, 123 . . . 128. In general, one or more of the contacts 120 are used to provide signals, and another one or more of the contacts 120 provide a signal return path. Accordingly, the lead body 111 can be used to deliver monopolar modulation (e.g., if the return contact is spaced apart significantly from the delivery contact), or bipolar modulation (e.g., if the return contact is positioned close to the delivery contact and in particular, at the same target neural population as the delivery contact). In still further embodiments, the pulse generator 101 (FIG. 1A) can operate as a return contact for monopolar modulation.

### 4.0 REPRESENTATIVE ELECTRICAL SIGNALS

FIG. 3A is a partially schematic illustration of a representative electrical therapy signal 300a used to delivery therapy in accordance with embodiments of the present technology. The therapy signal 300a includes biphasic pulses 301a repeating in a continuous manner. Each individual pulse 301a includes an anodic pulse phase 302a, a cathodic pulse phase 304a, and an interphase interval 306a separating the anodic pulse phase 302a and the cathodic pulse phase 304a. In the illustrated embodiment, the anodic pulse phase 302a and the cathodic pulse phase 304a are symmetrical (e.g., having generally equal pulse widths and generally equal and opposite amplitudes) such that individual pulses 301a are charge balanced. Individual pulses 301a are separated by an interpulse interval 308a. Together, the pulse 301a and the interpulse interval 308a define a pulse period 310a. The pulse period 310a repeats in cycles that define a frequency of the therapy signal 300a.

The therapy signal 300a can have relatively long pulse widths, such as between about 5 milliseconds and about 2 seconds. Accordingly, the anodic pulse phase 302a and the cathodic pulse phase 304a can each have a pulse width in a range of from about 5 milliseconds to about 2 seconds. In embodiments for which the pulse 301a is a monophasic pulse, the monophasic pulse phase can have a pulse width between about 5 milliseconds to about 2 seconds. In some embodiments, the therapy signal 300a can have a pulse width of between about 5 milliseconds and about 1 second, or between about 100 milliseconds and about 1 second, or between about 100 milliseconds and about 500 milliseconds, or between about 150 milliseconds and 1 second, or between about 150 milliseconds and about 750 milliseconds, or between about 150 milliseconds and about 500 milliseconds, or between about 200 milliseconds and about 1 second, or between about 200 milliseconds and about 750 milliseconds, or between about 200 milliseconds and about 500 milliseconds. Representative pulse widths include about 5 milliseconds, about 10 milliseconds, about 25 milliseconds, about 50 milliseconds, about 75 milliseconds, about 100 milliseconds, about 150 milliseconds, about 200 milliseconds, about 250 milliseconds, about 300 milliseconds, about 350 milliseconds, about 400 milliseconds, about 450 milliseconds, about 500 milliseconds, about 550 milliseconds, about 600 milliseconds, about 650 milliseconds, about 700 milliseconds, about 750 milliseconds, about 800 milliseconds, about 850 milliseconds, about 900 milliseconds, about 950 milliseconds, about 1 second, and/or about 2 seconds. In some embodiments, the pulse width is greater than about 5 milliseconds, greater than about 10 milliseconds, greater than about 25 milliseconds, greater than about 50 milliseconds, greater than about 75 milliseconds, greater than about 100 milliseconds, greater than about 200 milliseconds, greater than about 300 milliseconds, greater than about 400 milliseconds, greater than about 500 milliseconds, and/or greater than about 1 second.

In the illustrated embodiment, the anodic pulse phase 302a and the cathodic pulse phase 304a have generally equal pulse widths that can offset charge build up in a signal delivery element (e.g., electrodes 120) and/or surrounding tissue. In other embodiments, and as described below with respect to FIG. 3B, the anodic pulse phase and cathodic pulse phase do not have the same pulse width. In yet other embodiments, the stimulation charge recovery is a passive process, in which a shunt resistance is connected across the active electrodes to allow for charge built up on the output and Helmholtz capacitances from the therapeutic pulse (e.g., the anodic pulse phase 302a) to ‘bleed off’. In such embodiments, the therapy signal may be essentially a monophasic signal. As one skilled in the art will recognize, the frequency of the therapy signal 300a is based at least in part on the pulse width of the pulses 301a. For example, pulses with longer pulse widths typically (but not always) have lower frequencies. Accordingly, in some embodiments the frequency of the therapy signal 300a is less than about 100 Hz, less than about 10 Hz, less than about 5 Hz, and/or less than about 1 Hz.

The pulses 301a can have an amplitude (e.g., current amplitude or voltage amplitude) below the activation threshold of a target neuronal population. In such embodiments, the therapy signal 300a generally does not induce an action potential in target neurons when it is delivered to the target neuronal population, and therefore generally does not generate paresthesia in the patient. Generally, the activation of neurons depends on two variables: the strength (e.g., amplitude) of the signal and the duration (e.g., pulse width) for which the signal is applied. As the duration of the signal increases, the amplitude required to induce neuronal activation decreases. Accordingly, the amplitude of the pulses 301a is inversely related to the pulse width of the pulses 301a. In some embodiments, the amplitude remains below the rheobase of the target neuronal population. The rheobase refers to the minimum amplitude that results in neuronal activation when the therapy signal is applied for a continuous period (e.g., a period exceeding 100 milliseconds, 200 milliseconds, 300 milliseconds, etc.). In some embodiments, the rheobase can be approximated by measuring the amplitude at which a patient exhibits the first clinically discernable effects of the signal. For example, in some embodiments, the amplitude of the pulses 301a is about 3 mA or less, such as between about 0.1 mA and about 2.5 mA or between about 0.5 mA and about 2 mA.

Use of relatively long pulse widths such as those described for the signal 300a shown in FIG. 3A can cause substantial charge to build up in the electrodes delivering the signal. Therefore, the pulse width and the amplitude of the pulse 301a can also be selected to remain below an acceptable charge and/or charge density for the electrode materials used to deliver the therapy signal 300a. As one skilled in the art will appreciate, exceeding the acceptable or “maximum” charge and/or charge density of the electrodes may cause electrolysis on the surface of the electrode, distort the therapy signal delivered by the electrode, increase corrosion of the electrode, shorten the expected lifespan of the electrode, and/or cause the electrode to emit materials or products that can damage the surrounding tissue. Accordingly, the pulse width and amplitude of the pulse 301a can be selected to deliver a therapeutically effective charge to the target neural population without causing one or more of the foregoing events associated with exceeding a maximum charge density of the electrode. Suitable electrode materials include platinum and/or platinum alloys (e.g., platinum iridium) and/or other materials and alloys known in the art. For platinum electrodes, the pulse width and/or amplitude can be selected such that the charge density remains at or below about 300 μC/cm², which is currently a clinically acceptable maximum charge density for platinum electrodes. Table 1 below provides amplitude values that, for a set of particular pulse widths, generate a charge density of approximately 300 μC/cm² on a typical neurostimulation lead with electrodes of geometric surface area of approximately 12.7 mm².

TABLE 1
Signal Parameters for 300 μC/cm² Charge Density
on an Electrode with Geometric Surface Area of 12.7 mm²

| Pulse Width (ms) | Amplitude (mA) |
|---|---|
| 10 | 3.81 |
| 30 | 1.27 |
| 100 | 0.38 |
| 300 | 0.13 |
| 1000 | 0.04 |

The amplitude values in Table 1 are normalized to produce a 300 μC/cm² charge density at the recited pulse widths. The recited amplitude values therefore represent the “maximum” amplitude that, for the recited pulse widths, do not exceed the 300 μC/cm² charge density threshold. In embodiments in which the upper charge density threshold is a value other than 300 μC/cm², the corresponding amplitude values would change as well (assuming pulse width stays the same). For example, if the upper charge density threshold was greater than 300 μC/cm², the associated amplitude values would increase as well. In some embodiments, such as those described below with respect to FIGS. 5A and 5B, the maximum amplitude and/or upper charge density threshold that can be accommodated before inducing an event associated with exceeding a maximum charge density threshold for an electrode (e.g., waveform distortion, electrode surface bubbling, etc.) can be determined and used as the maximum amplitude and/or charge density threshold during waveform parameter selection.

In some embodiments, the pulse width and/or amplitude are selected such that the charge density approaches the maximum charge density permitted by the electrode material (e.g., within 5% of the maximum charge density, within 10% of the maximum charge density, within 20% of the maximum charge density, etc.). As described below, and without being bound by theory, applying the therapy signal 300a at an amplitude that is below the activation threshold of a target neuronal population but at an amplitude and pulse width combination that delivers relatively high charge densities is expected to directly suppress at least a subset of the target neuronal population. Because the native charge densities of polished electrodes can deliver sufficient charge over the relatively long pulse widths described herein, the electrodes do not necessarily require a coating material. However, in some embodiments, the electrodes nevertheless include a coating material (e.g., to increase the electrode surface area). The number of electrodes programmed to deliver the signal can also affect the total maximum charge. For example, in some embodiments, three electrodes are programmed as anodic and three electrodes are programmed as cathodic such that the total charge being delivered to the target neural population can be increased without exceeding the maximum charge density for any individual electrode.

In some embodiments, systems in accordance with the present technology include an algorithm that limits the stimulation charge to be below the acceptable charge and charge density for the electrode materials. Before delivering the therapy signal 300a, a user can input electrode information into a patient treatment system component (e.g., a graphical user interface on a modulator, controller, programmer, or other suitable device). In some embodiments, the electrode information is already stored in the patient treatment system, and/or the patient treatment system automatically calculates some or all of the electrode information. The electrode information can contain the electrode material, the surface area of the electrodes, and/or the number of electrodes (e.g., the number of anodes and the number of cathodes). In some embodiments, the surface area of the electrodes can be estimated from an impedance value associated with the electrode, which may be automatically detected by the system. For example, lower impedance values are associated with higher electrode surface areas. The user can then select a desired pulse width from a list or range of available pulse widths (e.g., between 5 milliseconds and 2 seconds). Based at least in part on the selected pulse width and the electrode information, the algorithm can determine the upper limit of the programmable amplitude (e.g., the “maximum amplitude”) based upon a calculation of maximum allowed charge density. For example, if a user inputs and/or the system determines that the electrodes are polished platinum iridium electrodes, the electrodes have a specific impedance value and/or surface area, and the pulse width is 30 milliseconds, the algorithm can calculate the maximum amplitude that can be used without exceeding a charge density of 300 μC/cm², which as reflected in Table 1 is about 1.27 mA.

In some embodiments, the maximum amplitude is below an activation threshold of a target neuronal population for the selected pulse width. For example, the maximum amplitude can be set below the rheobase of the first clinically discernable effect of the stimulation. As described in greater detail with respect to FIG. 4, the activation may be determined or approximated by slowly increasing the stimulation amplitude at a set pulse width (e.g., 5 milliseconds) and asking the patient to report any sensory or physical perceptions from the stimulation. If the activation threshold has an amplitude value less than the maximum amplitude based on the charge density calculation, the activation threshold amplitude value can be entered into the patient treatment system (e.g., via the interface on the modulator, controller, or programmer) to set a new maximum amplitude for the therapy signal 300a.

FIG. 3B is a partially schematic illustration of another representative therapy signal 300b. Certain aspects of therapy signal 300b are generally similar to those described above with respect to the therapy signal 300a shown in FIG. 3A. For example, therapy signal 300b includes a pulse period 310b having a biphasic pulse 301b and an interpulse interval 308b. The pulse 301b has an anodic pulse phase 302b and a cathodic pulse phase 304b separated by an interphase interval 306b. Unlike the therapy signal 300a shown in FIG. 3A, however, the pulses 301b shown in FIG. 3B do not have symmetrical anodic pulse phases 302b and cathodic pulse phases 304b. Rather, the cathodic pulse phase 304b has a shorter pulse width and a greater amplitude than the anodic pulse phase 302b. In other embodiments, therapy signal 300b can have an anodic pulse phase 302b that has a shorter pulse width and a greater amplitude than the cathodic pulse phase 304b (e.g., a mirror image of therapy signal 300b). Regardless, the pulse width and amplitude of the cathodic pulse phase 304b can nevertheless be selected such that the total charge delivered in the anodic pulse phase 302b and the cathodic pulse phase 304b remains substantially equal to avoid having a charge build up in the electrode or the patient's tissue. In some embodiments, the amplitude of the anodic pulse phase 302b can remain below the activation threshold that results in the first clinically discernable effect of the stimulation (and/or below the rheobase). Similarly, the larger amplitude of the cathodic pulse phase 302b also remains below the threshold of a clinically discernable effect of stimulation (and/or below the rheobase). One expected advantage of therapy signal 300b is the recovery period takes less time, meaning the duration between subsequent anodic pulse phases 302b is less and the frequency of the pulse period 310b can be higher.

FIG. 3C illustrates a representative therapy signal 300c with a ramping period 312. The ramping period 312 transitions between a maximum amplitude of the anodic pulse phase 302c and a maximum amplitude of the cathodic pulse phase 304c. In some embodiments (e.g., as described below with respect to FIG. 3E), the anodic pulse phase 302c and the cathodic pulse phase 304c can include a portion of the ramping period 312. The ramping period 312 can have a duration that is substantially equal to the pulse width of the therapy signal 300c. For example, if the anodic pulse phase 302c and the cathodic pulse phase 304c each have a pulse width of about 100 milliseconds, the ramping period 312 can have a duration of about 100 milliseconds. Accordingly, the ramping period 312 can have a duration between about 5 milliseconds and about 2 seconds. In other embodiments, the ramping period 312 is less than or greater than the pulse width of the anodic pulse phase 302c and/or the cathodic pulse phase 304c. Although the anodic pulse phase 302c and the cathodic pulse phase 304c are illustrated as symmetrical, the anodic pulse phase 302c and the cathodic pulse phase 304c can also have a configuration similar to that described above with respect to FIG. 3B, with the ramping period 312 extending therebetween.

FIG. 3D illustrates another therapy signal 300d having a non-continuous ramping period 312 between the anodic pulse phase 302d and the cathodic pulse phase 304d during pulse period 310d. The noncontinuous ramping period can include a first ramping period 312a immediately following the anodic pulse phase 302d and the cathodic pulse phase 304d, and a second ramping period 312b immediately preceding the anodic pulse phase 302d and the cathodic pulse phase 304d. The first ramping period 312a and the second ramping period 312b can be separated by an interphase interval 306d (e.g., between an anodic pulse phase 302d and cathodic pulse phase 304d within the same pulse 301d) or an interpulse interval 308d (e.g., between adjacent pulses 301d). The first ramping period 312a and the second ramping period 312b can have the same or different durations. Together, the first ramping period 312a and the second ramping period 312b can have a duration substantially equal to the pulse width of the therapy signal 301d (e.g., between about 5 milliseconds and about 2 seconds). In other embodiments, the first ramping period 312a and the second ramping period 312b together have a duration that is less than or greater than the pulse width of the therapy signal 301d.

As indicated above, some embodiments of the present technology include therapy signals having ramped, or at least partially ramped, anodic pulse phases and/or ramped, or at least partially ramped, cathodic pulse phases. For example, FIG. 3E illustrates a representative ramped therapy signal 300e in which the therapy signal 300e includes an anodic pulse phase 302e having a first ramping period 312a and a second ramping period 312b, and a cathodic pulse phase 304e having a third ramping period 312c and a fourth ramping period 312d. As described above, the anodic pulse phase 302e may have a pulse width of between about 5 milliseconds and about 2 seconds, and the cathodic pulse phase 304e may have a pulse width of between about 5 milliseconds and about 2 seconds. The second ramping period 312b can immediately follow the first ramping period 312a, as illustrated in FIG. 3E, or the second ramping period 312b can be spaced apart from the first ramping period 312a by a period of the anodic pulse phase 302a having a constant amplitude (e.g., as illustrated in FIG. 3C). Likewise, the fourth ramping period 312d can immediately follow the third ramping period 312c, as also illustrated in FIG. 3E, or the fourth ramping period 312d can be spaced apart from the third ramping period 312c by a period of the cathodic pulse phase 304a having a constant amplitude (e.g., as illustrated in FIG. 3C). In the illustrated embodiment, the second ramping phase 312b of the anodic pulse phase 302e immediately transitions into the third ramping period 312c of the cathodic pulse phase 304e. However, in other embodiments the second ramping period 312b of the anodic pulse phase 302e can be separated from the third ramping period 312c of the cathodic pulse phase 304e by an interphase interval (e.g., as illustrated in FIG. 3D). In the illustrated embodiment, the pulse period 310e is equal to the duration of the pulse 301e. However, in other embodiments respective pulses 301e can be separated by an interpulse interval (e.g., the fourth ramping period 312d can be separated from the first ramping period 312a).

FIG. 3F illustrates another representative therapy signal 300f used to deliver therapy in accordance with embodiments of the present technology. Unlike the therapy signals 300a-300e of FIGS. 3A-3E, which have a square wave form, a ramped wave form, or a combination thereof, the therapy signal 300f of FIG. 3F has a sinusoidal wave form pattern (or other non-linear pattern) comprising repeating curved pulses 301f. Each individual pulse 301f has an anodic pulse phase 302f and a cathodic pulse phase 304f. Similar to the signals 300a-300e described above, the anodic pulse phase 302f may have a pulse width between about 5 milliseconds and about 2 seconds, and the cathodic pulse phase 304f may have a pulse width between about 5 milliseconds and about 2 seconds.

Any of the signals 300a-f described above may further include offset high frequency pulses and/or bursts of high frequency pulses occurring during the anodic and/or cathodic pulse phases (e.g., pulses starting and ending at the non-zero amplitudes of the anodic and cathodic pulse phases). For example, FIG. 3G illustrates a representative signal 300g that is generally similar to the therapy signal 300a shown in FIG. 3A but further includes high frequency pulses 316g occurring during the anodic pulse phase 302g and the cathodic pulse phase 304g of the pulse 301g. As illustrated, the high frequency pulses 316g generally occur during the anodic pulse phase 302g and/or the cathodic pulse phase 304g, but are generally absent during the interphase interval 306g and the interpulse interval 308g. Although shown as occurring during both the anodic pulse phase 302g and the cathodic pulse phase 304g, in other embodiments, the high frequency pulses 316g occur only during the anodic pulse phase 302g. In yet other embodiments, the high frequency pulses 316g occur only during the cathodic pulse phase 304g.

The high frequency pulses 316g can have a frequency in a frequency range of from about 1.2 kHz and about 100 KHz. For example, the high frequency pulses 316g can have a frequency in a frequency range of from about 1.2 kHz to about 50 kHz, from about 1.2 KHz to about 25 kHz, from about 3 kHz to about 15 kHz, or from about 5 kHz to about 15 KHz. In some embodiments, the high frequency pulses 316g have a frequency of about 5 kHz, about 10 KHz, about 15 kHz, about 20 KHz, about 25 kHz, about 50 KHz, or about 100 kHz. The high frequency pulses 316g can have a pulse width in a pulse width range of from about 10 microseconds to about 333 microseconds, from about 25 microseconds to about 166 microseconds, from about 33 microseconds to about 100 microseconds, or from about 50 microseconds to about 166 microseconds. In some embodiments, such as the embodiment illustrated in FIG. 3G, the high frequency pulses 316g have an amplitude that is greater than the amplitude of the underlying anodic pulse phase 302g and/or the cathodic pulse phase 304g. In other embodiments, the high frequency pulses 316g have an amplitude that is equal to or less than the amplitude of the anodic pulse phase 302g and/or the cathodic pulse phase 304g. Further yet, although shown as bi-phasic pulses, the high frequency pulses 316g may instead be monophasic pulses. The high frequency pulses 316g can also have a ramped and/or sinusoidal shape.

The anodic pulse phase 302g may have an overall pulse width between about 5 milliseconds and about 2 seconds (not accounting for any phase change during the high frequency pulses 316g), as described in detail above for the signal 300a of FIG. 3A. Likewise, the cathodic pulse phase 304g may also have an overall pulse width between about 5 milliseconds and about 2 seconds (not accounting for any phase change during the high frequency pulses 316g).

The therapy signal 300g can be described as having a base component (e.g., base component pulses or low frequency pulses having the anodic pulse phase 302g and the cathodic pulse phase 304g) and a high frequency component (e.g., the high frequency pulses 316g). The base component may also be referred to as a low frequency component. In some embodiments, the base component and the high frequency component are a single waveform, and therefore are generally administered using the same electrodes/contacts. In other embodiments, the high frequency component is superimposed over the base component to create the therapy signal 300g.

FIG. 3H illustrates another representative signal 300h used to deliver therapy in accordance with embodiments of the present technology. The signal 300h is generally similar to the signal 300g shown in FIG. 3G, but instead of delivering high frequency pulses during the entirety of (or at least approximately the entirety of) the anodic pulse phase and the cathodic pulse phase, the signal 300h includes high frequency pulse bursts 314h (“the bursts 314h”) occurring during only portions of the anodic pulse phase 302h and the cathodic pulse phase 304h of the pulse 301h. Sequential bursts 314h are separated by a quiescent period 318h. As illustrated, the bursts 314h generally occur during the anodic pulse phase 302h and/or the cathodic pulse phase 304h, but are generally absent during the interphase interval 306h and the interpulse interval 308h. Although shown as occurring during both the anodic pulse phase 302h and the cathodic pulse phase 304h, in other embodiments, the bursts 314h occur only during the anodic pulse phase 302h. In yet other embodiments, the bursts 314h occur only during the cathodic pulse phase 304h.

The bursts 314h include one or more individual high frequency pulses 316h repeating at an intra-burst frequency in a frequency range of from about 1.2 kHz and about 100 kHz, from about 1.2 kHz to about 50 kHz, from about 1.2 kHz to about 25 kHz, from about 3 kHz to about 15 kHz, or from about 5 kHz to about 15 kHz. In some embodiments, the intra-burst frequency of the high frequency pulses 316h is about 5 kHz, about 10 KHz, about 15 kHz, about 20 kHz, about 25 kHz, about 50 kHz, or about 100 KHz. The high frequency pulses 316h can have a pulse width in a pulse width range of from about 10 microseconds to about 333 microseconds, from about 25 microseconds to about 166 microseconds, from about 33 microseconds to about 100 microseconds, or from about 50 microseconds to about 166 microseconds. In some embodiments, such as the embodiment illustrated in FIG. 3H, the high frequency pulses 316h have an amplitude that is greater than the amplitude of the underlying anodic pulse phase 302h and/or the cathodic pulse phase 304h. In other embodiments, the high frequency pulses 316h have an amplitude that is equal to or less than the amplitude of the anodic pulse phase 302h and/or the cathodic pulse phase 304h. Further yet, although shown as bi-phasic pulses, the high frequency pulses 316h may instead be monophasic pulses. The high frequency pulses 316h can also have a ramped and/or sinusoidal shape.

Each sequential burst of the high frequency pulse bursts 314h can include the same or a different number of individual high frequency pulses 316h, compared to the preceding burst 314h. For example, in the illustrated embodiment, the bursts 314h are shown as having either eight or four individual high frequency pulses 316h. In other embodiments, other numbers of high frequency pulses 316h can be delivered during the bursts 314h.

The anodic pulse phase 302h may have an overall pulse width between about 5 milliseconds and about 2 seconds (not accounting for any phase change during the bursts 314h), as described in detail above for the signal 300a of FIG. 3A. Likewise, the cathodic pulse phase 304h may also have an overall pulse width between about 5 milliseconds and about 2 seconds (not accounting for any phase change during the bursts 314h).

As with the therapy signal 300g of FIG. 3G, the therapy signal 300h of FIG. 3H can also be described as having a base component (e.g., the anodic pulse phase 302h and the cathodic pulse phase 304h) and a high frequency component (e.g., burst 314h of high frequency pulses 316h). In some embodiments, the base component and the high frequency component are a single waveform. In other embodiments, the high frequency component is superimposed over the base component to create the therapy signal 300h.

In some embodiments, the electrical signals 300a-h shown and described with reference to FIGS. 3A-3H do not produce paresthesia when delivered to the patient, and can therefore be referred to as “non-paresthesia producing electrical signals” or “paresthesia-free electrical signals.” For example, the electrical signals 300a-h can be delivered at an amplitude below an activation threshold of certain neurons, and/or below a perception threshold of the patient. This is independent of whether the patient is suffering from neuropathy-induced paresthesia (e.g., paresthesia or tingling caused by a neuropathy as opposed to paresthesia induced by the electrical signal). Accordingly, in some embodiments a paresthesia-free signal may be administered to a patient suffering from neuropathy induced paresthesia. Indeed, as set forth throughout this Detailed Description, in some embodiments the electrical signals provided herein can even reduce neuropathy-induced paresthesia. Thus, in some embodiments, the electrical signals (1) do not induce paresthesia in the patient, and (2) reduce naturally occurring paresthesia.

In operation, one or more of the signals 300a-h shown and described with reference to FIGS. 3A-3H can be generated by a signal generator (e.g., the signal generator 101 of FIG. 1A) and delivered to a patient's spinal cord region via an implanted signal delivery device (e.g., the signal delivery elements 110 of FIG. 1A) to treat the patient's neuropathy. Without intending to be bound by theory, the signals 300a-h described above may treat one or more aspects of the patient's neuropathy, including pain, sensation loss, impaired proprioception, and/or abnormal sensations such as neuropathy induced paresthesia. For example, the signals 300a-h may improve or otherwise normalize the afferent transmission of external sensory input to the patient's sensory cortex, thereby improving the patient's proprioception and the patient's ability to perceive such external sensory inputs. At the same time, the signals 300a-h may suppress or otherwise normalize neural activity in the dorsal horn pain circuits, thereby reducing the patient's inherent pain and/or abnormal sensations. In this way, the signals 300a-h are expected to beneficially address multiple aspects of the patient's neuropathy (e.g., the signals 300a-h may both reduce the patient's neuropathic pain while simultaneously restoring sensation).

As a result, and without being bound by theory, the electrical signals 300a-h shown and described with reference to FIGS. 3A-3H are expected to be useful in treating various neuropathies, including diabetic neuropathy, chemotherapy induced neuropathy, idiopathic neuropathy, peripheral polyneuropathy, and the like. In such embodiments, the reduction in patient pain and numbness is expected to provide clinical benefits beyond just alleviating the patient's pain and numbness. For example, neuropathy patients with reduced numbness/improved sensation are less likely to unknowingly injure themselves, which in neuropathy patients with impaired sensation can often lead to downstream consequences including infection and amputation. Improving the patient's ability to sense external sensory stimuli is expected to reduce the likelihood of such events. Similarly, neuropathy patients with reduced numbness and pain and improved proprioception are likely to be more active, which in turn can have positive effects on other manifestations of the patient's neuropathy and/or underlying conditions (e.g., Type-2 Diabetes).

The assignee of the present application, Nevro Corp., has conducted preliminary human clinical trials to further assess the effectiveness of using spinal cord stimulation with signal delivery parameters similar to those described herein to treat patients suffering from sensation loss, among other ailments. For example, a first patient presented with chronic low back pain, left lower limb pain, and partial loss of sensation in the left lower limb. An electrical signal having a pulse width of 900 milliseconds and an amplitude of 1 mA was delivered to the patient's spinal cord. The spinal cord stimulation both relieved the patient's back and limb pain and restored sensation in the patient's limb. Another patient similarly received pain relief and restoration of sensation bilaterally in both lower limbs following delivery of an electrical signal having a pulse width of 500 milliseconds and an amplitude of 1 mA to the patient's spinal cord. Yet another patient received bilateral pain relief and bilateral sensory restoration in the lower limbs following delivery of an electrical signal having a pulse width of 900 milliseconds and an amplitude of 1 mA to the patient's spinal cord.

### 5.0 REPRESENTATIVE METHODS FOR DETERMINING SUITABLE AMPLITUDES OF ELECTRICAL SIGNALS HAVING LONG PULSE WIDTHS

FIG. 4 is a block diagram illustrating a method 400 for treating a patient in accordance with embodiments of the present technology. Some or all of the steps in the method 400 can be performed by a processor executing instructions stored on or more elements of a patient treatment system. The method 400 can include receiving a first input including electrode information (step 402). The electrode information can relate to certain characteristics of one or more electrodes of the patient treatment system that are implanted in, or implantable into, the patient for delivering an electrical signal to a target neural population. In particular, the electrode information can include, among other things, the electrode material, the surface area of the electrode, and/or the number of electrodes. In addition to or in lieu of the surface area, the electrode information can include impedance values associated with the one or more electrodes. If impedance values are received, the method 400 can optionally include calculating the surface area based at least in part on the impedance values. The first input can be received from a user inputting the electrode information into a graphical user interface or other suitable means included as part of the patient treatment system, such as a modulator, controller, programmer, or other suitable device. In some embodiments, the first input can be received by accessing a memory storing the electrode information. In addition, one or more of the foregoing inputs can be received/retrieved from a computer-readable storage medium, and/or otherwise generated without a direct user input.

The method 400 can continue by receiving a second input corresponding to a desired pulse width (step 404). The second input can also be received from a user inputting the electrode information into the graphical user interface or other suitable device. In some embodiments, the graphical user interface can include a list of available pulse widths (e.g., ranging from 5 milliseconds to 2 seconds) and the input corresponds to a user selecting one of the available pulse widths. In other embodiments, the user can directly input a desired pulse width without selecting from a list of available pulse widths. In yet other embodiments, the patient treatment system may automatically select or recommend a pulse width.

Based at least in part on the electrode information and the pulse width, the processor can calculate a maximum amplitude that can be delivered without exceeding the maximum charge density of the electrodes and/or inducing events associated with exceeding the maximum charge density of the electrodes (e.g., using the algorithm described above with respect to FIG. 3A) (step 406). In some embodiments, the maximum amplitude can be determined by testing the waveform having the selected pulse width at incremental amplitudes until an event associated with exceeding the maximum charge density of the electrodes is observed, as described later with respect to FIGS. 5A and 5B. The maximum amplitude may also be set to avoid exciting the target neuronal tissue (e.g., the maximum amplitude can be below an activation threshold of the neurons). To determine the maximum amplitude that avoids exciting the target neuronal tissue, a signal with the selected pulse width can be delivered to the patient via the electrodes, and the amplitude can be increased (e.g., incrementally or continuously) until either (i) the maximum amplitude based on the charge density calculation is reached, or (ii) a clinically discernable effect other than the therapeutic effect of the therapy signal is observed in the patient. If the maximum amplitude based on the charge density calculation is reached before a clinically discernable effect other than the therapeutic effect of the therapy signal is observed, the maximum amplitude remains unchanged. If, however, the amplitude at which the clinically discernable effect is observed is less than the maximum amplitude based on the charge density calculation, the amplitude at which the clinically discernable effect is observed can be set as the new maximum amplitude. In other embodiments, the amplitude at which the clinically discernable effect is observed can be determined before calculating the maximum amplitude based on the charge density calculation.

The method 400 can continue by directing a therapy signal having the pulse width and an amplitude less than or equal to the maximum amplitude to the target neural population (step 408). For example, the modulator, controller, or programmer can direct a pulse generator to generate the therapy signal and deliver, via the electrodes, the therapy signal to the target neural population. Without being bound by theory, the therapy signal can treat the patient's neuropathy (e.g., diabetic neuropathy) by restoring lost sensation and/or reducing pain or other abnormal sensations.

Other suitable methods for delivering the therapy signals described herein can also be used. In some embodiments, the steps of receiving the electrode information and determining a maximum amplitude based on the maximum charge density can be omitted. In such embodiments, a pulse width is selected and various amplitudes are tested to determine a maximum amplitude beyond which the patient begins to exhibit a clinically discernable effect. The signal can then be applied at an amplitude less than the determined maximum amplitude. The therapy signals described herein can also be applied in combination with other therapies, such as high frequency SCS or conventional SCS.

As indicated above, the present technology further includes techniques for determining a maximum amplitude that can be delivered at a given pulse width. For example, FIG. 5A is a flowchart of a method 500 for determining a maximum amplitude at which a waveform having a given pulse width can be administered, without distorting the waveform. The method can begin in step 502 by recording the voltage potential of an electrode outputting a waveform having a given pulse width at a plurality of incremental amplitudes. This may include selecting a pulse width (e.g., 10 microseconds, 30 microseconds, 100 microseconds, 300 microseconds, 1,000 microseconds, etc.) and recording a trace (e.g., a measurement of the electrode voltage over time, as might be observed on an oscilloscope trace) of the waveform having the selected pulse width at the plurality of incremental amplitudes. The increment may include a stepwise increment, with each subsequent amplitude increasing by a common amount (e.g., 100 uA) relative to a preceding amplitude. In other embodiments, the increment between subsequent amplitudes may be variable. FIG. 5B provides an example of a plurality of voltage potential traces recorded using a voltage measurement system (in this case, an oscilloscope) for a waveform having a pulse width of 300 microseconds. The scope traces were recorded at incremental amplitudes of 50 μA, 100 μA, 150 μA, 200 μA, 300 μA, 400 μA, 500 μA, 750 μA, and 1,000 μA. In some embodiments, the voltage can be measured at step 502 on the surface of the electrode being tested, via another electrode positioned adjacent the electrode being tested, via a resistor connected in series between a pulse generator and the electrode being tested, or another suitable recording technique.

Returning to FIG. 5A, the method 500 can continue in step 504 by determining, based on the recordings taken in step 502, the amplitude at which the waveform begins to distort. This may include, for example, manually or automatically analyzing the morphology of the voltage potential or other recordings measured in step 502 to identify waveform distortion. In some embodiments, waveform distortion may present as a reversal or inflection in the slope of the waveform in the voltage potential as the voltage potential increases (e.g., which may be referred to as a “shoulder”). The amplitude at which the shoulder or inflection appears is therefore the amplitude at which waveform distortion begins. In FIG. 5B, for example, the shoulder (identified by the arrows marked “x”) is first observed in the voltage potential trace of the waveform at an amplitude of 200 μA. In some embodiments, determining the amplitude at which the waveform distorts can include identifying a reversal or inflection in the slope that exceeds a minimum threshold to account for any potential noise in the signal.

The method 500 can continue in step 506 by determining (e.g., selecting) the maximum amplitude for the waveform based at least in part on the determination made in step 504. For example, the maximum amplitude can be set as the largest tested amplitude that does not induce waveform distortion. In FIG. 5B, for example, the largest tested amplitude at which waveform distortion does not occur is 150 μA. Thus, 150 μA could be selected as the maximum amplitude. Optionally, determining the maximum amplitude can include testing a plurality of additional incremental amplitudes between the amplitude determined in step 504 and the amplitude determined in step 506 to further “fine tune” the maximum amplitude. For example, in the embodiment shown in FIG. 5B, steps 502, 504, and 506 could be repeated for a plurality of incremental amplitudes between 150 μA and 200 μA.

Once the maximum amplitude is determined in step 506, the method 500 can optionally continue in step 508 by calculating the maximum charge density based on the maximum amplitude determined in step 504. This may be done by multiplying the pulse width and the amplitude to obtain the charge per phase of the waveform, and then dividing the charge per phase by the electrode surface area.

In some embodiments, steps 502-506 of the method 500 can be performed with the electrode in a saline bath or other suitable environment rather than with the electrode implanted in a patient. In other embodiments, steps 502-506 can be performed during or after the lead is introduced into the body (e.g., with the lead outside of the epidural space within the epidural space). In such embodiments, the testing can be brief to minimize hydrolysis and other electrochemical reactions. Regardless, once the maximum amplitude is determined in step 506 and/or the maximum charge density is determined in step 508, the method 500 can continue in step 510 by programming a signal generator to deliver a therapy signal having an amplitude less than or equal to the maximum amplitude to a target neural population in a patient's spinal cord region.

Of note, waveform distortion is often the first adverse effect of exceeding a maximum charge density threshold of an electrode. Thus, while other adverse effects (e.g., electrode bubbling, electrode corrosion, etc.) can occur if the maximum charge density is exceeded, such events usually occur at amplitudes that are greater than the amplitude at which waveform distortion occurs. Thus, using waveform distortion to set the maximum amplitude threshold is also expected to prevent the other adverse effects identified herein that are associated with exceeding a maximum charge density of an electrode.

### 6.0 REPRESENTATIVE EXAMPLES

The following examples are provided to further illustrate embodiments of the present technology and are not to be interpreted as limiting the scope of the present technology. To the extent that certain embodiments or features thereof are mentioned, it is merely for purposes of illustration and, unless otherwise specified, is not intended to limit the present technology. It will be understood that many variations can be made in the procedures described herein while still remaining within the bounds of the present technology. Such variations are intended to be included within the scope of the presently disclosed technology.
1. A method of treating a patient having diabetic neuropathy, the method comprising:
   programming a signal generator to deliver an electrical signal having a pulse width in a pulse width range of from about 5 milliseconds to about 2 seconds to the patient's spinal cord region via an implantable signal delivery device,
   wherein the electrical signal restores sensation lost in association with the diabetic neuropathy, reduces patient pain associated with the diabetic neuropathy, and/or reduces abnormal patient sensations associated with the diabetic neuropathy.
2. The method of example 1 wherein programming the signal generator is done in response to the patient having diabetic neuropathy.
3. The method of example 1 or example 2 wherein the electrical signal restores sensation lost in association with the diabetic neuropathy.
4. The method of example 3 wherein the electrical signal restores sensation lost in the patient's hands and/or feet.
5. The method of example 3 or example 4 wherein programming the signal generator is done in response to the patient having sensation loss associated with diabetic neuropathy.
6. The method of example 1 or example 2 wherein the electrical signal reduces patient pain associated with the diabetic neuropathy.
7. The method of example 6 wherein the electrical signal reduces pain in the patient's hands and/or feet.
8. The method of example 6 or example 7 wherein programming the signal generator is done in response to the patient having pain associated with the diabetic neuropathy.
9. The method of example 1 or example 2 wherein the electrical signal reduces abnormal patient sensations associated with the diabetic neuropathy.
10. The method of example 9 wherein the abnormal patient sensations include neuropathy-induced paresthesia in the patient's hands and/or feet.
11. The method of example 9 or example 10 wherein programming the signal generator is done in response to the patient having abnormal sensations associated with the diabetic neuropathy.
12. The method of any of examples 1-11 wherein the pulse width range is from about 5 milliseconds to about 1 second.
13. The method of any of examples 1-11 wherein the pulse width range is from about 100 milliseconds to about 1 second.
14. The method of any of examples 1-11 wherein the pulse width range is from about 100 milliseconds to about 500 milliseconds.
15. The method of any of examples 1-14 wherein the electrical signal does not induce paresthesia in the patient.
16. The method of any of examples 1-15 wherein the electrical signal includes a plurality of biphasic pulses having an anodic pulse phase and a cathodic pulse phase, and wherein at least one of the anodic pulse phase or the cathodic pulse phase has the pulse width in the pulse width range from about 5 milliseconds to about 2 seconds.
17. The method of example 16 wherein the anodic pulse phase and the cathodic pulse phase have different pulse widths and/or amplitudes.
18. The method of example 16 wherein the anodic pulse phase and the cathodic pulse phase have the same pulse width and amplitude.
19. The method of any of examples 1-15 wherein the electrical signal includes a plurality of monophasic pulses, and wherein the plurality of monophasic pulses have the pulse width in the pulse width range of from about 5 milliseconds to about 2 seconds.
20. The method of any of examples 1-19 wherein the diabetic neuropathy is painful diabetic neuropathy.
21. The method of any of examples 1-19 wherein the diabetic neuropathy is painless diabetic neuropathy.
22. A patient treatment system for treating diabetic neuropathy, the system comprising:
   an implantable signal delivery device postionable proximate a patient's spinal cord region; and
   a signal generator having a controller programmed with instructions that, when executed, cause the signal generator to:
      deliver an electrical signal to the patient's spinal cord region via the implantable signal delivery device,
      wherein the electrical signal has a pulse width in a pulse width range of from about 5 milliseconds to about 2 seconds, and
      wherein the electrical signal restores sensation lost in association with the diabetic neuropathy, reduces patient pain associated with the diabetic neuropathy, and/or reduces abnormal patient sensations associated with the diabetic neuropathy.
23. The system of example 22 wherein the electrical signal restores sensation and reduces patient pain.
24. The system of example 22 wherein the electrical signal restores sensation and reduces abnormal patient sensations.
25. The system of any of examples 22-24 wherein the pulse width range is from about 100 milliseconds to about 1 second.
26. The system of any of examples 22-24 wherein the pulse width range if from about 150 milliseconds to about 750 milliseconds.
27. The system of any of examples 22-26 wherein the electrical signal is a paresthesia-free electrical signal.
28. The system of any of examples 22-27 wherein the signal generator is an implantable signal generator.
29. The system of any of examples 22-28 wherein the implantable signal delivery device is positionable within an epidural space of the patient's spinal cord region.
30. A method of treating diabetic neuropathy in a patient, the method comprising:
   delivering an electrical signal to the patient's spinal cord region via an implanted signal delivery device,
   wherein the electrical signal has a pulse width in a pulse width range of from about 5 milliseconds to about 2 seconds, and
   wherein the electrical signal restores sensation lost in association with the diabetic neuropathy, reduces patient pain associated with the diabetic neuropathy, and/or reduces abnormal patient sensations associated with the diabetic neuropathy.
31. The method of example 30 wherein the electrical signal restores sensation.
32. The method of example 30 or example 31 wherein the electrical signal restores sensation and reduces patient pain.
33. The method of any of examples 30-32 wherein the pulse width range is from about 200 milliseconds to about 750 milliseconds.
34. The method of any of examples 30-33 wherein the electrical signal does not induce paresthesia in the patient.
35. A method of treating a patient having peripheral neuropathy, the method comprising:
   programming a signal generator to deliver an electrical signal having a pulse width in a pulse width range of from about 5 milliseconds to about 2 seconds to the patient's spinal cord region via an implantable signal delivery device,
   wherein the electrical signal restores sensation lost in association with the peripheral neuropathy.
36. The method of example 35 wherein the electrical signal reduces abnormal patient sensations in addition to restoring lost sensation.
37. The method of example 35 wherein the electrical signal reduces patient pain in addition to restoring lost sensation.
38. The method of any of examples 35-37 wherein the pulse width range is from about 150 milliseconds to about 1 second.
39. The method of any of examples 35-37 wherein the pulse width range is from about 200 milliseconds to about 500 milliseconds.
40. The method of any of any of examples 35-39 wherein the electrical signal does not induce paresthesia in the patient.
41. The method of any of examples 35-40 wherein the peripheral neuropathy includes diabetic neuropathy.
42. The method of any of examples 35-40 wherein the peripheral neuropathy includes chemotherapy induced neuropathy.
43. The method of any of examples 35-40 wherein the peripheral neuropathy includes idiopathic neuropathy.

### 7.0 CONCLUSION

From the foregoing, it will be appreciated that specific embodiments of the disclosed technology have been described herein for purposes of illustration, but that various modifications may be made without deviating from the technology. For example, therapy signals described herein can be delivered at combinations of parameter values within the foregoing ranges at values that are not expressly disclosed herein. Certain aspects of the technology described in the context of particular embodiments may be combined or eliminated in other embodiments. For example, the therapy signal can be monophasic with a passive charge elimination phase. In some embodiments, the foregoing techniques can be used to address patient deficits other than sensation loss, pain, or abnormal sensations. Further, while advantages associated with certain embodiments of the disclosed technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

The use of “and/or,” as in “A and/or B” refers to A alone, B alone, and both A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Where a range of values is provided, it is understood that each intervening value, to the tenth of the unit of the lower limit unless the context clearly dictates otherwise, to between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Each of the patents and patent application publications referenced herein are incorporated by reference in their entireties and for all purposes. However, to the extent any patent or patent application publication conflicts with the present disclosure, the present disclosure controls.

I/We claim:

1. A method of treating a patient having diabetic neuropathy, the method comprising:
programming a signal generator to deliver an electrical signal having a pulse width in a pulse width range of from about 5 milliseconds to about 2 seconds to the patient's spinal cord region via an implantable signal delivery device,
wherein the electrical signal restores sensation lost in association with the diabetic neuropathy, reduces patient pain associated with the diabetic neuropathy, and/or reduces abnormal patient sensations associated with the diabetic neuropathy.

2. The method of claim 1 wherein programming the signal generator is done in response to the patient having diabetic neuropathy.

3. The method of claim 1 wherein the electrical signal restores sensation lost in association with the diabetic neuropathy.

4. The method of claim 3 wherein the electrical signal restores sensation lost in the patient's hands and/or feet.

5. The method of claim 3 wherein programming the signal generator is done in response to the patient having sensation loss associated with diabetic neuropathy.

6. The method of claim 1 wherein the electrical signal reduces patient pain associated with the diabetic neuropathy.

7. The method of claim 6 wherein the electrical signal reduces pain in the patient's hands and/or feet.

8. The method of claim 6 wherein programming the signal generator is done in response to the patient having pain associated with the diabetic neuropathy.

9. The method of claim 1 wherein the electrical signal reduces abnormal patient sensations associated with the diabetic neuropathy.

10. The method of claim 9 wherein the abnormal patient sensations include neuropathy-induced paresthesia in the patient's hands and/or feet.

11. The method of claim 9 wherein programming the signal generator is done in response to the patient having abnormal sensations associated with the diabetic neuropathy.

12. The method of claim 1 wherein the pulse width range is from about 5 milliseconds to about 1 second.

13. The method of claim 1 wherein the pulse width range is from about 100 milliseconds to about 1 second.

14. The method of claim 1 wherein the pulse width range is from about 100 milliseconds to about 500 milliseconds.

15. The method of claim 1 wherein the electrical signal does not induce paresthesia in the patient.

16. The method of claim 1 wherein the electrical signal includes a plurality of biphasic pulses having an anodic pulse phase and a cathodic pulse phase, and wherein at least one of the anodic pulse phase or the cathodic pulse phase has the pulse width in the pulse width range from about 5 milliseconds to about 2 seconds.

17. The method of claim 16 wherein the anodic pulse phase and the cathodic pulse phase have different pulse widths and/or amplitudes.

18. The method of claim 16 wherein the anodic pulse phase and the cathodic pulse phase have the same pulse width and amplitude.

19. The method of claim 1 wherein the electrical signal includes a plurality of monophasic pulses, and wherein the plurality of monophasic pulses have the pulse width in the pulse width range of from about 5 milliseconds to about 2 seconds.

20. The method of claim 1 wherein the diabetic neuropathy is painful diabetic neuropathy.

21. The method of claim 1 wherein the diabetic neuropathy is painless diabetic neuropathy.

22. A patient treatment system for treating diabetic neuropathy, the system comprising:
an implantable signal delivery device postionable proximate a patient's spinal cord region; and
a signal generator having a controller programmed with instructions that, when executed, cause the signal generator to:
deliver an electrical signal to the patient's spinal cord region via the implantable signal delivery device,
wherein the electrical signal has a pulse width in a pulse width range of from about 5 milliseconds to about 2 seconds, and
wherein the electrical signal restores sensation lost in association with the diabetic neuropathy, reduces patient pain associated with the diabetic neuropathy, and/or reduces abnormal patient sensations associated with the diabetic neuropathy.

23. The system of claim 22 wherein the electrical signal restores sensation and reduces patient pain.

24. The system of claim 22 wherein the electrical signal restores sensation and reduces abnormal patient sensations.

25. The system of claim 22 wherein the pulse width range is from about 100 milliseconds to about 1 second.

26. The system of claim 22 wherein the pulse width range if from about 150 milliseconds to about 750 milliseconds.

27. The system of claim 22 wherein the electrical signal is a paresthesia-free electrical signal.

28. The system of claim 22 wherein the signal generator is an implantable signal generator.

29. The system of claim 22 wherein the implantable signal delivery device is positionable within an epidural space of the patient's spinal cord region.

30. A method of treating diabetic neuropathy in a patient, the method comprising:
delivering an electrical signal to the patient's spinal cord region via an implanted signal delivery device,
wherein the electrical signal has a pulse width in a pulse width range of from about 5 milliseconds to about 2 seconds, and
wherein the electrical signal restores sensation lost in association with the diabetic neuropathy, reduces patient pain associated with the diabetic neuropathy, and/or reduces abnormal patient sensations associated with the diabetic neuropathy.

31. The method of claim 30 wherein the electrical signal restores sensation.

32. The method of claim 30 wherein the electrical signal restores sensation and reduces patient pain.

33. The method of claim 30 wherein the pulse width range is from about 200 milliseconds to about 750 milliseconds.

34. The method of claim 30 wherein the electrical signal does not induce paresthesia in the patient.

35. A method of treating a patient having peripheral neuropathy, the method comprising:
programming a signal generator to deliver an electrical signal having a pulse width in a pulse width range of from about 5 milliseconds to about 2 seconds to the patient's spinal cord region via an implantable signal delivery device,
wherein the electrical signal restores sensation lost in association with the peripheral neuropathy.

36. The method of claim 35 wherein the electrical signal reduces abnormal patient sensations in addition to restoring lost sensation.

37. The method of claim 35 wherein the electrical signal reduces patient pain in addition to restoring lost sensation.

38. The method of claim 35 wherein the pulse width range is from about 150 milliseconds to about 1 second.

39. The method of claim 35 wherein the pulse width range is from about 200 milliseconds to about 500 milliseconds.

40. The method of claim 35 wherein the electrical signal does not induce paresthesia in the patient.

41. The method of claim 35 wherein the peripheral neuropathy includes diabetic neuropathy.

42. The method of claim 35 wherein the peripheral neuropathy includes chemotherapy induced neuropathy.

43. The method of claim 35 wherein the peripheral neuropathy includes idiopathic neuropathy.
